(19) United States
(12) Patent Application Publication
(10) Pub. No.: US 2026/0265548 A1
(43) Pub. Date: Sep. 10, 2026

(54) COMPOSITIONS AND METHODS FOR MICROBIOLOGICAL CONTROL IN A PAPER MACHINE COATING KITCHEN

(71) Applicant: ECOLAB USA Inc., St. Paul, MN (US)

(72) Inventors: Bjorn KAMLIN, Storvreta (SE); Arie Michiel POOS, Bergschenhoek (NL); Jean-Michel MAQUINGHEN, Leiden (NL); Laura E. RICE, St. Charles, IL (US); Markus REHMONEN, Naantali (FI); Bert SIMONS, Amstelveen (NL)

(73) Assignee: ECOLAB USA Inc., St. Paul, MN (US)

(21) Appl. No.: 19/552,383

(22) Filed: Feb. 27, 2026

Related U.S. Application Data
(60) Provisional application No. 63/767,465, filed on Mar. 5, 2025.

(51) Int. Cl. C09D 7/61; A01N 59/00; A01P 1/00; C12Q 3/00; G05D 21/02

(52) U.S. Cl. CPC C09D 7/61; A01N 59/00; A01P 1/00; C12Q 3/00; G05D 21/02

(57) ABSTRACT

The present disclosure provides compositions and methods for controlling microbiological activity. A method for controlling microbiological activity in a coating slurry and/or a component thereof may include adding a stabilizer, a chlorine source, and water to a reactor, mixing the stabilizer, the chlorine source, and the water in the reactor to form a stabilized chlorine composition, measuring a first pH of the coating slurry and/or the component thereof with a pH sensor and/or measuring a first oxidation-reduction potential (ORP) of the coating slurry and/or the component thereof with an ORP sensor, adding the stabilized chlorine composition to the coating slurry and/or the component thereof via a delivery conduit, and adding additional stabilized chlorine composition to the coating slurry and/or the component thereof via the delivery conduit if the measured first pH and/or first ORP decreases.

### TECHNICAL FIELD

The present disclosure generally relates to microbiological control. More particularly, the disclosure relates to microbiological control in paper coating slurries and/or components thereof using stabilized chlorine compositions.

### BACKGROUND

Biological/microbiological activity in process streams and/or process slurries is problematic for a variety of reasons and can lead to problems with sanitation, process equipment efficiency, and product quality. For example, in papermaking processes, high biological activity levels have a deleterious effect on equipment operation.

Oxidant biocides, such as peroxide acid and sodium hypochlorite, have been widely used in the pulp and paper industry. These oxidant biocides are highly effective at immediately killing large numbers of microorganisms. Unfortunately, after their introduction into process water systems, oxidant biocides are unstable and they tend to oxidize rapidly and lose their effectiveness. As such, sufficient numbers of microorganisms may survive an oxidant biocide treatment and repeated applications of oxidant biocide is typically not commercially feasible and can have deleterious effects on paper brighteners, dyes, and other additives required to produce commercially acceptable paper products. Repeated introduction of oxidant biocides can also corrode many pieces of papermaking machinery. One technique used to address this problem is to stabilize the oxidant biocides, thereby allowing them to suppress the viability of microorganisms over longer periods of time while weakening the negative impact that the oxidant biocides have on the resulting paper and the papermaking equipment.

In a paper coating process, multiple components (e.g., dyes, pigments, clays, etc.) may be dosed into a mixing tank to form a coating slurry. Each component may be present in an aqueous medium before addition to the mixing tank and the final coating slurry may also comprise an aqueous medium. The level of microbiological activity in the aqueous mediums needs to be controlled or the coating process will fail.

### BRIEF SUMMARY

The present disclosure provides compositions and methods for controlling microbiological activity.

In some embodiments, the disclosure provides a method for controlling microbiological activity in a coating slurry and/or a component thereof. The method comprises adding a stabilizer, a chlorine source, and water to a reactor; mixing the stabilizer, the chlorine source, and the water in the reactor to form a stabilized chlorine composition; measuring a first pH of the coating slurry and/or the component thereof with a pH sensor and/or measuring a first oxidation-reduction potential (ORP) of the coating slurry and/or the component thereof with an ORP sensor; adding the stabilized chlorine composition to the coating slurry and/or the component thereof via a delivery conduit; and adding additional stabilized chlorine composition to the coating slurry and/or the component thereof via the delivery conduit if the measured first pH and/or first ORP decreases.

In some embodiments, the present disclosure provides a method of monitoring and controlling microbiological activity in a coating slurry and/or a component thereof. The method comprises (a) providing a monitoring and controlling unit comprising a controller and a plurality of sensors in communication with the controller, wherein the plurality of sensors includes a pH sensor operable to measure a pH of the coating slurry and/or the component thereof and an ORP sensor operable to measure an ORP of the coating slurry and/or the component thereof; (b) entering an acceptable range for the pH of the coating slurry and/or the component thereof and the ORP of the coating slurry and/or the component thereof into the controller; (c) adding a stabilizer, a chlorine source, and water to a reactor;
(d) mixing the stabilizer, the chlorine source, and the water in the reactor; (e) forming a stabilized chlorine composition in the reactor; (f) adding the stabilized chlorine composition to the coating slurry and/or the component thereof via a delivery conduit; (g) transporting a portion of the coating slurry and/or the component thereof via a side stream conduit to an inlet of the monitoring and controlling unit; (h) measuring the pH and the ORP of the coating slurry and/or the component thereof with the pH sensor and the ORP sensor, respectively; (i) determining if the measured pH is within the acceptable range entered into the controller in step (b); (j) determining if the measured ORP is within the acceptable range entered into the controller in step (b); (k) carrying out a corrective action if the measured pH and/or ORP is outside of the acceptable range entered into the controller in step (b); and (l) optionally repeating one or more of steps (a) to (k) to determine if the pH and/or ORP of the coating slurry and/or the component thereof has been brought within the acceptable range entered in step (b).

The foregoing has outlined rather broadly the features and technical advantages of the present disclosure in order that the detailed description that follows may be better understood. Additional features and advantages of the disclosure will be described hereinafter that form the subject of the claims of this application.

### DETAILED DESCRIPTION

Various embodiments are described below. The relationship and functioning of the various elements of the embodiments will be better understood in light of the following detailed description. However, elements and embodiments are not strictly limited to those explicitly described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

The terms “polymer,” “copolymer,” “polymerize,” “copolymerize,” and the like include not only polymers comprising two monomer residues and polymerization of two different monomers together, but also include (co) polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, organic, inorganic, and/or functionally modified. A polymer of the present disclosure can be in the form of a solution, a dry powder, a liquid, or a dispersion, for example.

The paper manufacturing process can be organized into different general sections. For example, one section includes the location where a pulp furnish is disposed as a thin layer on a moving papermaking wire or forming fabric. Another section is commonly referred to as the “press section,” which is where the thin layer is pressed to remove additional water. Following the press section is the dryer section where the pressed layer moves through a series of heated rollers. At this point, the dry substrate can optionally be rewetted by passing it through a size press and further dried by passing it through another set of heated rollers. Finally, the dried substrate passes through a paper finishing section, such as a calendaring section. The dried substrate may also or alternatively be transported to a coating section, where a coating may be applied to one or both sides of the paper sheet (see, for example, Handbook for Pulp and Paper Technologists, 3rd Edition, by Gary A. Smook, Angus Wilde Publications Inc., (2002) and The Nalco Water Handbook (3rd Edition), by Daniel Flynn, McGraw Hill (2009)). The compositions and methods disclosed herein can be incorporated into or carried out in any of the foregoing sections.

The present disclosure provides compositions and methods for controlling microbiological activity in various aqueous mediums, such as a coating slurry or a component of a coating slurry. A component of a coating slurry includes any aqueous medium containing one or more components that may be added to a coating slurry or used to prepare a coating slurry.

For example, a component of a coating slurry may include a latex, a dye, an optical brightener, a starch, pigment, a thickener, a biocide, a defoamer, a base (e.g., NaOH), or any combination thereof. In certain instances, the component of a coating slurry may comprise water that is to be subsequently combined with an additional component, such as an optical brightening agent, a stabilizer, etc.

In some embodiments, the component of the coating slurry comprises a calcium carbonate slurry, a clay slurry, a starch slurry, a paint slurry, and any combination thereof.

In certain embodiments, the coating slurry and/or the component thereof further comprises a latex, a dye, an optical brightener, or any combination thereof.

One or more coating slurries may be used to coat a paper sheet and each of the coating slurries may be treated with the compositions and methods disclosed herein. In addition to treating the coating slurries, the present disclosure also contemplates treating one or more of the various raw materials/components used to prepare the coating slurries. Treating the raw materials/components with the compositions disclosed herein is useful to prevent contamination of the coating slurry, which makes preservation of the coating slurry more cost effective and reduces the need for biocide.

Compositions of the present disclosure may be produced by adding a stabilizer, a chlorine source, and water to a reactor. A base, such as sodium hydroxide, may optionally be added to the reactor. Alternatively or additionally, the stabilizer and the chlorine source may be added directly to the coating slurry and/or the component thereof. In the reactor, slurry, and/or component thereof, the stabilizer, the chlorine source, and the water are mixed to form a stabilized chlorine composition. The stabilized chlorine composition may comprise, for example, monochloramine.

The stabilizer is not particularly limited and may be selected from, for example, an ammonium salt, ammonium chloride, ammonium sulphate, ammonium bromide, ammonium carbamate, ammonium hydroxide, ammonia, urea, dimethylhydantoin, and any combination thereof.

In some embodiments, the stabilizer comprises an ammonium source. In certain embodiments, the stabilizer comprises a single species of stabilizer. For example, while various prior art methods may use multiple/different species of stabilizers to stabilize chlorine, various embodiments of the present disclosure only need a single species of stabilizer to stabilize the chlorine.

The chlorine source is not particularly limited and may be selected from, for example, a hypochlorite (e.g., sodium hypochlorite), hypochloric acid, electrochemically generated chlorine, and any combination thereof.

The amount of chlorine to stabilizer in the stabilized chlorine composition may be considered. In illustrative embodiments, the stabilized chlorine composition may comprise a weight ratio of chlorine to stabilizer of about 1:1 to about 1:30, such as about 1:1 to about 1:20, about 1:1 to about 1:10, about 1:1 to about 1:5, about 1:1 to about 1:2.5, about 1:1 to about 1:2, about 1:1.1, about 1:1.2, about 1:1.3, about 1:1.4, about 1:1.5, about 1:1.6, about 1:1.7, about 1:1.8, or about 1:1.9.

The present inventors determined that excess chlorine has detrimental impacts on the methods and compositions disclosed herein. The inventors also determined that a substantial excess of stabilizer may also have detrimental impacts on the compositions and methods disclosed herein. In some embodiments, a stabilized chlorine composition of the present disclosure comprises from about 1 ppm to about 10,000 ppm of the chlorine, such as about 100 ppm to about 9,000 ppm, about 500 ppm to about 8,000 ppm, about 1,000 ppm to about 7,000 ppm, about 2,000 ppm to about 6,000 ppm, about 3,000 ppm to about 6,000 ppm, about 4,000 ppm to about 6,000 ppm, or about 5,000 ppm to about 6,000 ppm.

The target dosing concentration depends upon the characteristics of the particular coating slurry and/or component thereof. For example, dosing may be impacted by oxidant demand and/or the level of microbiological contamination. Illustrative, non-limiting examples of dosing protocols can be seen in Table 1 wherein a dose of 10 ppm may be appropriate for a “clean” coating and/or component thereof and a dose of 100 ppm may be appropriate for a slurry and/or component thereof with elevated levels of microbiological contamination.

TABLE 1

| | | | L stabilized Cl2 |
|---|---|---|---|
| | Conc. | Dosing | added to achieve |
| Tank size | Stabilized | conc | target dosing |
| (L) | Cl2 (ppm) | (ppm) | concentration |
| 1,000 | 5000 | 10 | 2 |
| | 5000 | 100 | 20 |
| 10,000 | 5000 | 10 | 20 |
| | 5000 | 100 | 200 |
| 100,000 | 5000 | 10 | 200 |
| | 5000 | 100 | 2000 |

In an additional illustrative example, about 1.6 L of a chlorine source may be mixed with about 1 L of a ~20 wt. % stabilizer solution to form a mixture. The mixture may be further diluted with water, if desired. For example, the mixture may be diluted by about 5, 10, 15, 20, or 25 times with water.

The pH of the stabilized chlorine composition may be from, for example, about 6 to about 14, such as about 7 to about 14, about 7.5 to about 14, about 8 to about 14, about 8.5 to about 14, about 9 to about 14, about 9.5 to about 14, or about 10 to about 14.

In a method for controlling microbiological activity in a coating slurry and/or a component thereof, a first pH of the coating slurry and/or component thereof may be measured with a pH sensor. Additionally or alternatively, a first oxidation-reduction potential (ORP) of the coating slurry and/or the component thereof may be measured with an ORP sensor. The first pH and the first ORP are recorded and then subsequent pH and ORP measurements are carried out on a continuous or intermittent basis. If subsequent pH and/or ORP measurements drift too far from the first pH and/or ORP measurements, certain corrective actions may be taken.

A stabilized chlorine composition of the present disclosure may be added to the coating slurry and/or the component thereof via a delivery conduit. The delivery conduit may be disposed between the reactor, a storage tank, chest, a mixing tank, and/or any other device comprising the stabilized chlorine composition and the tank, chest, etc., comprising the coating slurry and/or the component thereof.

In certain embodiments, while carrying out subsequent pH and/or ORP measurements, if decreases in pH and/or ORP are detected, additional amounts of stabilized chlorine may be added to the coating slurry and/or component thereof. Additional amounts may be added in a variety of ways. For instance, after an initial amount of stabilized chlorine is added and a decrease in pH and/or ORP is detected, any additional amount of stabilized chlorine may be added until the pH and/or ORP have been raised back into the appropriate ranges.

In some embodiments, if the pH drops by about 0.5 pH units (e.g., to about 6.5 from an initial reading of about 7.0), a corrective action may be taken, such as adding additional stabilized chlorine to the coating slurry and/or component thereof.

In some embodiments, if the first ORP decreases by about 50 mV (e.g., from about 150 mV to about 100 mV), a corrective action may be taken, such as adding additional stabilized chlorine to the coating slurry and/or component thereof.

Additional stabilized chlorine may be added gradually over a period of time (such as a few minutes to a few hours) or a high dose of stabilized chlorine may be added in a short period of time (such as a few seconds to a few minutes).

A coating slurry and/or component thereof typically has reduced amounts of water as water can be detrimental. Accordingly, when adding stabilizer, chlorine source, stabilized chlorine, etc., to the coating slurry and/or component thereof, it is desirable to minimize the amount of water being added.

Any medium, coating slurry, and/or component thereof may have various levels of water in accordance with the present disclosure. For example, a coating slurry or component thereof may have from about 1 wt. % to about 50 wt. % water, such as about 10 wt. % to about 50 wt. %, about 20 wt. % to about 50 wt. %, about 30 wt. % to about 50 wt. %, about 40 wt. % to about 50 wt. %, about 1 wt. % to about 40 wt. %, about 1 wt. % to about 30 wt. %, about 1 wt. % to about 20 wt. %, or about 1 wt. % to about 10 wt. %.

After addition of the stabilizer, chlorine source, and/or stabilized chlorine, the amount of water in the medium, coating slurry, and/or component thereof does not substantially increase. For example, in some embodiments, from about 0.1 wt. % to about 0.5 wt. % of water, such as about 0.4 wt. %, about 0.3 wt. %, or about 0.2 wt. % of water is added to the medium, coating slurry and/or component thereof with the stabilizer, the chlorine source, and/or the stabilized chlorine. For instance, if stabilized chlorine in 2 liters of water was added to 1,000 liters of a coating slurry, about 0.2 wt. % (based on weight of slurry) of water would be added.

In some embodiments, the pH sensor and/or the ORP sensor is submerged in the coating slurry and/or the component thereof. In certain embodiments, a skid comprises the pH sensor and/or the ORP sensor. When a skid comprises the pH and/or ORP sensor, a side stream conduit may transport a portion/sample of the coating slurry and/or the component thereof to the pH sensor and/or the ORP sensor. As will be further described below, the pH sensor and/or the ORP sensor may be in communication with a controller.

The delivery conduit, which may transport stabilizer, chlorine source, and/or stabilized chlorine from a reactor, storage tank, mixing tank, etc., to a coating slurry and/or component thereof, may be flushed with water after adding the stabilized chlorine, stabilizer, and/or chlorine source to the coating slurry and/or the component thereof. Moreover, if the stabilizer, water, and chlorine source are mixed in a reactor to form a stabilized chlorine composition and the composition is transported to, for example, a coating slurry, the reactor may be subsequently flushed with water before adding additional chlorine source and stabilizer back into the reactor to form additional stabilized chlorine.

During the flushing process, the end of the delivery conduit closest to the destination where the chemical is to be transported is sealed, such as by closing a valve, to prevent the contents of the delivery conduit from flowing into the destination vessel. A separate valve on the delivery conduit may be opened to allow the contents of the delivery conduit (e.g., stabilizer, chlorine source, stabilized chlorine, etc.) to be evacuated and transported to, for example, a drain or a storage vessel. If a liquid is used to flush the delivery conduit, the liquid may also be transported to the drain or storage vessel. In some embodiments, forced air or different type of gas may be pushed through the conduit to flush the contents.

The liquid used to flush the delivery conduit is not particularly limited. Illustrative, non-limiting examples of liquids that may be used include fresh water, municipal water, recycled water, surface water, condensed water, cooling water, ground water, or any mixture thereof. In some embodiments, the liquid may include an amount, such as from about 0.001 wt. % to about 5 wt. %, about 0.001 wt. % to about 3 wt. %, about 0.001 wt. % to about 1 wt. %, or about 0.001 wt. % to about 0.1 wt. % of an additional chemical, such as a stabilizer, a chlorine source, and/or a stabilized chlorine.

Minimizing water content in a coating slurry and/or a component thereof may be advantageous because higher water content can lead to poor coating quality (streaks, poor adhesion, etc.), increased drying time (slower production rates and increased energy demand), and a weaker coating surface (durability and performance, e.g., print quality).

Moreover, the reactor, storage tank, mixing tank, holding tank, or any other vessel comprising the stabilizer, chlorine source, and/or stabilized chlorine may include a delivery conduit for each different destination where the stabilizer, chlorine source, and/or stabilized chlorine is to be transported. In certain embodiments, the reactor, storage tank, mixing tank, holding tank, etc., may have from 1 to about 50 (or more) delivery conduits, such as from about 4 to about 40, about 4 to about 30, about 4 to about 20, about 6 to about 24, or any other number of delivery conduits necessary in order to transport the stabilizer, chlorine source, and/or stabilized chlorine to every different destination where it needs to be transported.

For example, if stabilized chlorine within a tank was to be transported to a first coating slurry, a second coating slurry, a calcium carbonate slurry, and a clay slurry, the tank may comprise a first delivery conduit to transport the stabilized chlorine to the first coating slurry, a second delivery conduit to transport the stabilized chlorine to the second coating slurry, a third delivery conduit to transport the stabilized chlorine to the calcium carbonate slurry, and a fourth delivery conduit to transport the stabilized chlorine to the clay slurry.

The tank comprising the delivery conduits is not particularly limited and may be, for example, the reactor where the chlorine source and stabilizer are mixed to form the stabilized chlorine, a mixing tank, a storage tank, a distribution tank, a holding tank, or any other vessel capable of containing the chemical (e.g., stabilizer, chlorine source, and/or stabilized chlorine) until it needs to be transported to a particular destination.

The stabilized chlorine could be transported to each destination at the same time or at different times. For example, stabilized chlorine could be transported to a first coating slurry at a first period of time, stabilized chlorine could be transported to a second coating slurry at a second period of time, such as about 30 minutes after the first period of time, stabilized chlorine could be transported to a clay slurry at a third period of time, such as about 30 minutes after the second period of time, etc.

Each delivery conduit may optionally comprise a valve located as close as possible (e.g., 1-5 inches, 6-12 inches, 12-24 inches, etc.) to the destination (e.g., the tank comprising the coating slurry). For instance, if a delivery conduit was 10 feet long, the valve could be placed about 6 inches from the end of the conduit connected to the destination (e.g., tank comprising the coating slurry) such that when the delivery conduit is flushed with water after delivery of stabilizer, chlorine source, and/or stabilized chlorine, only the contents of the 6 inches of conduit closest to the destination (i.e., downstream of the valve) will be transported into the destination instead of the contents of the entire length of the conduit.

While various embodiments of the present disclosure refer to adding a stabilizer and a chlorine source to a reactor and forming the stabilized chlorine in the reactor, the methods of the present disclosure can also be carried out in the absence of the reactor. For example, the stabilizer and the chlorine source can be added directly to a coating slurry and/or a component thereof.

Accordingly, a method for controlling microbiological activity in a coating slurry and/or a component thereof may comprise measuring a first pH of the coating slurry and/or the component thereof with a pH sensor and/or measuring a first oxidation-reduction potential (ORP) of the coating slurry and/or the component thereof with an ORP sensor. The first measured pH and the first measured ORP may serve as a baseline for the slurry and/or component thereof. Then, a stabilizer and a chlorine source may be added to the coating slurry and mixed to form a stabilized chlorine in the coating slurry and/or component thereof.

The pH and/or ORP may be continuously or intermittently monitored and if a decrease in pH and/or ORP is detected (as compared to the first measured pH and/or first measured ORP), additional stabilizer, chlorine source, and/or stabilized chlorine may be added to the coating slurry and/or the component thereof until the pH and/or ORP is raised back to the first measured pH and/or ORP value (or higher).

Any method disclosed herein may be carried out with an automated monitoring and controlling system, which may be used for measuring, controlling, and/or optimizing one or more system parameters or properties of a medium (e.g., ORP, pH, redox, tank level, etc.) in the process. Optimization can include, for example, measuring one or more properties associated with the medium to be sure that the one or more properties are within an acceptable, predetermined range and, if the one or more properties are not within the acceptable, predetermined range for each respective property being measured, causing a change in the medium to bring the property back within the acceptable, predetermined range.

In certain embodiments, the system includes a monitoring and controlling unit that comprises a controller and a plurality of sensors. Each of the plurality of sensors can be in communication with the controller. For example, if the unit comprises three sensors, each of the three sensors can be in communication with the controller. In certain aspects, the monitoring and controlling unit can be attached to a skid, or other type of support member, to allow for mobility.

As used herein, the term “controller” refers to a manual operator or an electronic device having components, such as a processor, memory device, digital storage medium, a communication interface including communication circuitry operable to support communications across any number of communication protocols and/or networks, a user interface (e.g., a graphical user interface that may include cathode ray tube, liquid crystal display, plasma display, touch screen, or other monitor), and/or other components.

The controller is preferably operable for integration with one or more application-specific integrated circuits, programs, computer-executable instructions or algorithms, one or more hard-wired devices, wireless devices, and/or one or more mechanical devices. Moreover, the controller is operable to integrate the feedback, feed-forward, and/or predictive loop(s) of the invention. Some or all of the controller system functions may be at a central location, such as a network server, for communication over a local area network, wide area network, wireless network, internet connection, microwave link, infrared link, wired network (e.g., Ethernet) and the like. In addition, other components, such as a signal conditioner or system monitor, may be included to facilitate signal transmission and signal-processing algorithms.

In certain aspects, the controller includes hierarchy logic to prioritize any measured or predicted properties associated with system parameters. For example, the controller may be programmed to prioritize system pH over ORP, or vice versa. It should be appreciated that the object of such hierarchy logic is to allow improved control over the system parameters and to avoid circular control loops.

In some embodiments, the monitoring and controlling unit and method associated therewith includes an automated controller. In some embodiments, the controller is manual or semi-manual. For example, when the system includes one or more datasets received from various sensors in the system, the controller may either automatically determine which data points/datasets to further process or an operator may partially or fully make such a determination. A dataset for an industrial body of water, for instance, may include variables or system parameters such as ORP, dissolved oxygen (DO), conductivity, pH, turbidity, and/or concentrations of certain chemicals, such as chlorine, biocides, etc. Such system parameters are typically measured with any type of suitable data capturing equipment, such as sensors designed specifically for these parameters, e.g., pH sensors, ORP sensors, ion analyzers, temperature sensors, thermocouples, pressure sensors, corrosion probes, and/or any other suitable device or sensor. Data capturing equipment is in communication with the controller and, according to some embodiments, may have advanced functions (including any part of the control algorithms described herein) imparted by the controller.

The monitoring and controlling unit may comprise a plurality of sensors, which are capable of analyzing the medium and transmitting data regarding the medium to the controller. The plurality of sensors may comprise, for example, sensors for measuring conductivity, pH, ORP, biocide concentration, turbidity, temperature, flow, and DO in the medium. The monitoring and controlling unit may comprise any of these sensors, all of these sensors, a combination of two or more of these sensors (e.g., pH and ORP), one or more additional sensors not specifically mentioned here, and the sensors may be in communication with the controller.

The monitoring and controlling unit may further comprise a fluorometer. The fluorometer may be configured to measure a fluorescent signal of a fluorescent tracer in the medium. If the medium comprises two or more tracers that are different, the fluorometer is capable of measuring a fluorescent signal of each different tracer.

The controller is capable of carrying out various corrective actions, such as sending a signal to a chemical injection pump causing the pump to add additional stabilized chlorine to a medium, such as a coating slurry, until the property (e.g., pH, ORP, etc.) is brought back within the predetermined acceptable range. A corrective action can also include, for example, sending a signal to a closed valve, causing it to open and allow chemical (e.g., stabilized chlorine) to flow from a storage device/vessel into the medium (e.g., coating slurry) or sending a signal to an open valve, causing it to close and stop the flow of chemical into the medium.

The presently disclosed monitoring and controlling system comprises, in certain embodiments, one or more chemical injection pumps and/or one or more valves. Each chemical injection pump and/or valve may be in fluid communication with a storage device. Each storage device may comprise one or more chemicals and the chemical injection pumps may transport those chemicals into the medium (e.g., coating slurry). A valve may block or allow the flow of the chemical into the medium. In some embodiments, the chemical injection pump comprises the storage device. The valve(s) and chemical injection pump(s) may be in communication with the controller in any number of ways, such as through any combination of wired connection, a wireless connection, electronically, cellularly, through infrared, satellite, or according to any other types of communication networks, topologies, protocols, standards and more. Accordingly, the controller can send signals to the valve(s) and/or pump(s) to control their chemical (e.g., rinse aid, detergent, etc.) feed rates.

In certain embodiments, the monitoring and controlling system is implemented to have the plurality of sensors, fluorometer, etc., provide continuous or intermittent feedback, feed-forward, and/or predictive information to the controller, which can relay this information to a relay device, such as the Nalco Global Gateway, which can transmit the information via cellular communications to a remote device, such as a cellular telephone, computer, and/or any other device that can receive cellular communications. This remote device can interpret the information and automatically send a signal (e.g. electronic instructions) back, through the relay device, to the controller to cause the controller to make certain adjustments to the output of the pumps and/or close or open a valve. The information can also be processed internally by the controller and the controller can automatically send a signal to the pump(s) to adjust the amount of chemical injection, for example, and/or send a signal to a valve(s) causing it to open or close (either fully or partially). Based upon the information received by the controller from the plurality of sensors, fluorometer, or from the remote device, the controller may transmit signals to the various pumps and/or valves to make automatic, real-time adjustments, to the amount of chemical that the pumps are injecting into the medium.

Alternatively, an operator of the remote device that receives cellular communications from the controller can manually manipulate the valves and/or pumps through the remote device. The operator may communicate instructions, through the remote device, cellularly or otherwise, to the controller and the controller can make adjustments to the valves and/or the rate of chemical addition of the chemical injection pumps.

For example, the operator can receive a signal or alarm from the remote device through a cellular communication from the controller and send instructions or a signal back to the controller using the remote device to turn on one or more of the chemical injection pumps, turn off one or more of the chemical injection pumps, increase or decrease the amount of chemical being added to the medium by one or more of the injection pumps, close a valve, open a valve, or any combination of the foregoing. The controller and/or the remote device is also capable of making any of the foregoing adjustments or modifications automatically without the operator sending or inputting any instructions.

Preset parameters or programs are entered into the controller or remote device so that the controller or remote device can determine if a measured property is outside of an acceptable range. Based on the information received by the plurality of sensors and/or fluorometer, the controller or remote device can make appropriate adjustments to the valves or pumps and/or send out an appropriate alert.

In certain embodiments, the remote device or controller can include appropriate software to receive data from the plurality of sensors and/or fluorometer and determine if the data indicates that one or more measured properties of the medium are within, or outside, an acceptable range. The software can also allow the controller or remote device to determine appropriate actions that should be taken to remedy the property that is outside of the acceptable range.

For example, if the measured pH is about 0.5 or more pH units below an initial pH reading for a medium, such as a coating slurry, the software allows the controller or remote device to make this determination and take remedial action, such as alerting a pump to increase the flow of stabilized chlorine into the coating slurry and/or opening a valve, thereby allowing stabilized chlorine to flow into the medium.

The monitoring and controlling system and/or controller disclosed herein can incorporate programming logic to convert analyzer signals from the plurality of sensors and/or fluorometer to pump adjustment logic and, in certain embodiments, control one or more of a valve(s) and/or a chemical injection pump(s) with a unique basis. Non-limiting, illustrative examples of the types of chemical injection pumps that can be manipulated include chemical injection pumps responsible for injecting stabilizers, chlorine sources, stabilized chlorine, and any other type of chemical that could prove to be useful in the particular medium.

The fluorometer and sensors disclosed herein are operable to sense and/or predict a property associated with the medium or system parameter and convert the property into an input signal, e.g., an electric signal, capable of being transmitted to the controller. A transmitter associated with the fluorometer and each sensor transmits the input signal to the controller. The controller is operable to receive the transmitted input signal, convert the received input signal into an input numerical value, analyze the input numerical value to determine if the input numerical value is within an acceptable range, generate an output numerical value, convert the output numerical value into an output signal, e.g., an electrical signal, and transmit the output signal to a receiver, such as a pump and/or valve incorporating such receiver capabilities or a remote device, such as a computer or cellular telephone, incorporating receiver capabilities. The receiver receives the output signal and either alerts an operator to make adjustments to the valve and/or the flow rates of the pump, or the receiver can be operable to cause a change in a valve (e.g., open it or close it) and/or in a flow rate of the pumps automatically, if the output numerical value is not within the acceptable range for that property.

The method is optionally repeated for a plurality of different system parameters, where each different system parameter has a unique associated property, or, alternatively, all system parameters can be analyzed concurrently by fluorometer and the plurality of sensors.

Data transmission of measured parameters or signals to chemical pumps, valves, alarms, remote monitoring devices, such as computers or cellular telephones, or other system components is accomplished using any suitable device, and across any number of wired and/or wireless networks, including as examples, WiFi, WiMAX, Ethernet, cable, digital subscriber line, Bluetooth, cellular technologies (e.g., 2G, 3G, Universal Mobile Telecommunications System (UMTS), GSM, Long Term Evolution (LTE), or more) etc. The Nalco Global Gateway is an example of a suitable device. Any suitable interface standard(s), such as an Ethernet interface, wireless interface (e.g., IEEE 802.11a/b/g/x, 802.16, Bluetooth, optical, infrared, radiofrequency, etc.), universal serial bus, telephone network, the like, and combinations of such interfaces/connections may be used.

As used herein, the term “network” encompasses all of these data transmission methods. Any of the described devices (e.g., archiving systems, data analysis stations, data capturing devices, process devices, remote monitoring devices, fluorometers, sensors, valves, chemical injection pumps, etc.) may be connected to one another using the above-described or other suitable interface or connection.

In some embodiments, system parameter information is received from the system and archived. In certain embodiments, system parameter information is processed according to a timetable or schedule. In some embodiments, system parameter information is immediately processed in real-time or substantially real-time. Such real-time reception may include, for example, “streaming data” over a computer network.

The chemicals to be added to the system, such as the stabilized chlorine, may be introduced to the medium using any suitable type of chemical injection pump. Most commonly, positive displacement injection pumps are used and are powered either electrically or pneumatically. Continuous flow injection pumps can also be used to ensure specialty chemicals are adequately and accurately injected into the medium. Though any suitable pump or delivery system may be used, exemplary pumps and pumping methods include those disclosed in U.S. Pat. No. 5,066,199, titled “Method for Injecting Treatment Chemicals Using a Constant Flow Positive Displacement Pumping Apparatus” and U.S. Pat. No. 5,195,879, titled “Improved Method for Injecting Treatment Chemicals Using a Constant Flow Positive Displacement Pumping Apparatus,” each incorporated herein by reference in its entirety.

In some embodiments, changes in the valves and/or chemical injection pumps are limited in frequency. In some aspects, adjustment limits are set at a maximum of 1 per 15 min and sequential adjustments in the same direction may not exceed 8, for example. In some embodiments, after 8 total adjustments or a change of 50% or 100%, the pump could be suspended for an amount of time (e.g., 2 or 4 hours) and alarm could be triggered. If such a situation is encountered, it is advantageous to trigger an alarm to alert an operator. Other limits, such as maximum pump output, may also be implemented. It should be appreciated that it is within the scope of the invention to cause any number of adjustments in any direction without limitation. Such limits are applied as determined by the operator or as preset into the controller.

In accordance with certain embodiments of the present disclosure, a method of monitoring and controlling one or more properties of a medium, such as a coating slurry, is provided. The properties can be, for example, pH and ORP.

The method includes the use of a monitoring and controlling unit comprising a controller, a pH sensor, and an ORP sensor. One or more pumps, which are in communication with the controller, are utilized to inject various chemicals, such as stabilized chlorine, a chlorine source, a stabilizer, etc., into the medium. Each chemical may have its own chemical injection pump. Also, each conduit responsible for transporting the chemical into the medium may comprise a valve, which may be opened or closed, to allow or block, respectively, the flow of the chemical into the medium.

An acceptable range for each of the one or more properties of the medium to be measured is entered into the controller. Alternatively, instead of entering an acceptable range, an acceptable lower limit could be entered and/or an acceptable upper limit. For example, the controller could be programmed such that additional stabilized chlorine is added if the pH drops below a certain level, such as 7. As an additional example, if the pH reading is high, such as 10, 11, 12, etc., the controller could be programmed to hold off on adding more stabilized chlorine to the medium until the pH drops below the acceptable lower limit.

A conduit may be provided between the medium and the monitoring and controlling unit. A sample of medium passes through the conduit and into an inlet of the monitoring and controlling unit. Next, one or more properties of the medium are measured using at least a pH sensor and/or an ORP sensor and the controller determines if the measured one or more properties are within the acceptable range entered into the controller in the previous step. This determining step can be automatically performed by the controller and in this step, the measured value for each measured property is compared to the acceptable range entered for that specific property.

If the measured one or more properties are outside of the acceptable range (or limit) associated with that property, the controller and/or operator of the controller may cause a change, for example, in an influx of a chemical into the medium from the one or more chemical injection pumps, the chemical(s) being capable of adjusting the measured property and bringing it back within the acceptable range. The controller is operable to determine when the measured property is back within the acceptable range and subsequently turn off the chemical injection pump(s).

In an illustrative, non-limiting embodiment, the present disclosure provides a method of monitoring and controlling microbiological activity in a coating slurry and/or a component thereof. The method comprises (a) providing a monitoring and controlling unit comprising a controller and a plurality of sensors in communication with the controller, wherein the plurality of sensors includes a pH sensor operable to measure a pH of the coating slurry and/or the component thereof and an ORP sensor operable to measure an ORP of the coating slurry and/or the component thereof; (b) entering an acceptable range for the pH of the coating slurry and/or the component thereof and the ORP of the coating slurry and/or the component thereof into the controller; (c) adding a stabilizer, a chlorine source, and water to a reactor; (d) mixing the stabilizer, the chlorine source, and the water in the reactor; (e) forming a stabilized chlorine composition in the reactor; (f) adding the stabilized chlorine composition to the coating slurry and/or the component thereof via a delivery conduit; (g) transporting a portion of the coating slurry and/or the component thereof via a side stream conduit to an inlet of the monitoring and controlling unit; (h) measuring the pH and the ORP of the coating slurry and/or the component thereof with the pH sensor and the ORP sensor, respectively; (i) determining if the measured pH is within the acceptable range entered into the controller in step (b); (j) determining if the measured ORP is within the acceptable range entered into the controller in step (b); (k) carrying out a corrective action if the measured pH and/or ORP is outside of the acceptable range entered into the controller in step (b); and (l) optionally repeating one or more of steps (a) to (k) to determine if the pH and/or ORP of the coating slurry and/or the component thereof has been brought within the acceptable range entered in step (b).

The acceptable range for the pH of the coating slurry and/or the component thereof is variable. For example, the pH may be between about 6 and about 11, such as about 7 to about 11, about 8 to about 11, about 9 to about 11, or about 10 to about 11.

The acceptable range for the ORP of the coating slurry and/or the component thereof is variable. For example, the ORP may be between about −200 mV and about 500 mV, such as from about −150 mV to about 400 mV, about −100 mV to about 300 mV, or about 0 mV to about 200 mV.

Various corrective actions may be carried out depending upon the nature of the irregularity. For example, a corrective action may include adding additional stabilized chlorine composition to the coating slurry and/or the component thereof, increasing an amount of time that the stabilized chlorine composition is added to the coating slurry and/or the component thereof, or a combination thereof.

Any of the methods disclosed herein may comprise or exclude a step of adding urea, sulfamic acid, ammonium sulfate, an organic biocide (e.g., DBNPA, glutaraldehyde), bromine, and/or bromide to the reactor, the coating slurry, and/or the component thereof.

Any composition disclosed herein may comprise or exclude urea, sulfamic acid, ammonium sulfate, an organic biocide (e.g., DBNPA, glutaraldehyde), bromine, and/or bromide.

The foregoing may be better understood by reference to the following examples, which are intended for illustrative purposes and are not intended to limit the scope of the disclosure or its application in any way.

### EXAMPLES

Freshly prepared monochloramine was tested against a 20 wt. % DBNPA product. Identical tests were performed in several different slurries. Some of the resulting data can be seen in Table 2.

TABLE 2

| | 10:20 | | | | Bacteria | cfu/ml | | | | |
|---|---|---|---|---|---|---|---|---|---|---|
| | ppm | | REDOX | pH | 1 h | 4 h | 24 h | REDOX | pH | |
| | 0 | P0 | 95 | 8.72 | 320,000 | 460,000 | 860,000 | 20 | 8.94 | Coating |
| MCA | 10 | P1 | N/A | N/A | 5,700 | 700 | 300 | 21 | 8.97 | Colour |
| | 20 | P2 | N/A | N/A | 400 | 100 | 300 | 23 | 8.93 | station |
| | 40 | P3 | N/A | N/A | 200 | 200 | 400 | 20 | 8.93 | |
| COMP 1 | 100 | P4 | N/A | N/A | 100,000 | 320,000 | 620,000 | 19 | 8.93 | |
| | 200 | P5 | N/A | N/A | 200 | 50,000 | 4,900 | 20 | [symbol] | |
| | 400 | P6 | N/A | N/A | 200 | [symbol] | 100 | 21 | [symbol] | |
| | 0 | K0 | 154 | 8.24 | 1,120,000 | 1,260,000 | 3,120,000 | 48 | 8.29 | Capim |
| MCA | 10 | K1 | N/A | N/A | 1,020,000 | 70,000 | 810,000 | 51 | 8.29 | Clay |
| | 20 | K2 | N/A | N/A | 2,700 | 800 | [symbol] | 57 | [symbol] | |
| | 40 | K3 | N/A | N/A | 600 | 200 | [symbol] | 172 | 8.27 | |
| COMP 1 | 100 | K4 | N/A | N/A | 1,200,000 | 90,000 | 580,000 | [symbol] | 8.26 | |
| | 200 | K5 | N/A | N/A | 360,000 | 30,000 | 1,200 | 73 | 8.24 | |
| | 400 | K6 | N/A | N/A | 1,700 | 900 | [symbol] | [symbol] | 8.28 | |

| | | ml |
|---|---|---|
| COMP 2 | hypo 15% | 6 |
| 9000 ppm | COMP 2 | 5 |
| | water | 90 |
| | pH | 9.4 |
| COMP 1 | COMP 1 | 1 |
| 1% | water | 99 |

[symbol]

indicates data missing or illegible when filed

As can be seen, the methods and compositions disclosed herein are useful for preventing or reducing an amount of microbiological contamination in a coating slurry and/or a component thereof. The presently disclosed stabilized chlorine compositions are effective for biocontrol and their performance is a competitive with non-oxidizing biocides.

In Table 2, “MCA” refers to monochloramine, “COMP 1” refers to a prior art non-oxidizing biocide, and “COMP 2” refers to a different prior art non-oxidizing biocide.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a stabilizer” is intended to include “at least one stabilizer” or “one or more stabilizers.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A method for controlling microbiological activity in a coating slurry and/or a component thereof, comprising:
adding a stabilizer, a chlorine source, and water to a reactor;
mixing the stabilizer, the chlorine source, and the water in the reactor to form a stabilized chlorine composition;
measuring a first pH of the coating slurry and/or the component thereof with a pH sensor and/or measuring a first oxidation-reduction potential (ORP) of the coating slurry and/or the component thereof with an ORP sensor;
adding the stabilized chlorine composition to the coating slurry and/or the component thereof via a delivery conduit; and
adding additional stabilized chlorine composition to the coating slurry and/or the component thereof via the delivery conduit if the measured first pH and/or first ORP decreases.

2. The method of claim 1, wherein from about 0.1 wt. % to about 0.5 wt. % of water is added to the coating slurry with the stabilized chlorine.

3. The method of claim 1, wherein the stabilizer is selected from the group consisting of an ammonium salt, ammonium chloride, ammonium sulphate, ammonium bromide, ammonium carbamate, ammonium hydroxide, ammonia, urea, dimethylhydantoin, and any combination thereof.

4. The method of claim 1, wherein the stabilizer comprises an ammonium source and/or a single species of stabilizer.

5. The method of claim 1, wherein the chlorine source is selected from the group consisting of a hypochlorite, hypochloric acid, electrochemically generated chlorine, and any combination thereof.

6. The method of claim 1, wherein the stabilized chlorine composition comprises a weight ratio of chlorine to stabilizer of about 1:1 to about 1:30.

7. The method of claim 1, wherein the stabilized chlorine comprises monochloramine.

8. The method of claim 1, further comprising adding a base to the reactor.

9. The method of claim 1, wherein the stabilized chlorine comprises a pH from about 7 to about 14.

10. The method of claim 1, wherein the pH sensor and/or the ORP sensor is submerged in the coating slurry and/or the component thereof.

11. The method of claim 1, wherein a skid comprises the pH sensor and/or ORP sensor.

12. The method of claim 1, further comprising adding additional stabilized chlorine to the coating slurry and/or the component thereof when the first pH decreases by about 0.5 pH units.

13. The method of claim 1, further comprising adding additional stabilized chlorine to the coating slurry and/or the component thereof when the first ORP decreases by about 50 mV.

14. The method of claim 1, further comprising flushing the delivery conduit with water after adding the stabilized chlorine to the coating slurry and/or the component thereof.

15. The method of claim 1, wherein the component of the coating slurry comprises a calcium carbonate slurry, a clay slurry, a starch slurry, a paint slurry, and any combination thereof.

16. The method of claim 1, further comprising flushing the reactor with water prior to adding the stabilizer and/or the chlorine source.

17. The method of claim 1, excluding adding urea, sulfamic acid, ammonium sulfate, an organic biocide, bromine, and/or bromide to the reactor, the coating slurry, and/or the component thereof.

18. A method of monitoring and controlling microbiological activity in a coating slurry and/or a component thereof, comprising:
(a) providing a monitoring and controlling unit comprising a controller and a plurality of sensors in communication with the controller, wherein the plurality of sensors includes a pH sensor operable to measure a pH of the coating slurry and/or the component thereof and an ORP sensor operable to measure an ORP of the coating slurry and/or the component thereof;
(b) entering an acceptable range for the pH of the coating slurry and/or the component thereof and the ORP of the coating slurry and/or the component thereof into the controller;
(c) adding a stabilizer, a chlorine source, and water to a reactor;
(d) mixing the stabilizer, the chlorine source, and the water in the reactor;
(e) forming a stabilized chlorine composition in the reactor;
(f) adding the stabilized chlorine composition to the coating slurry and/or the component thereof via a delivery conduit;
(g) transporting a portion of the coating slurry and/or the component thereof via a side stream conduit to an inlet of the monitoring and controlling unit;
(h) measuring the pH and the ORP of the coating slurry and/or the component thereof with the pH sensor and the ORP sensor, respectively;
(i) determining if the measured pH is within the acceptable range entered into the controller in step (b);
(j) determining if the measured ORP is within the acceptable range entered into the controller in step (b);
(k) carrying out a corrective action if the measured pH and/or ORP is outside of the acceptable range entered into the controller in step (b); and
(l) optionally repeating one or more of steps (a) to (k) to determine if the pH and/or ORP of the coating slurry and/or the component thereof has been brought within the acceptable range entered in step (b).

19. The method of claim 18, wherein the acceptable range for the pH of the coating slurry and/or the component thereof is from about 6 to about 11 and/or wherein the acceptable range for the ORP of the coating slurry and/or the component thereof is from about −200 mV to about 500 mV.

20. A method for controlling microbiological activity in a coating slurry and/or a component thereof, comprising:
measuring a first pH of the coating slurry and/or the component thereof with a pH sensor and/or measuring a first oxidation-reduction potential (ORP) of the coating slurry and/or the component thereof with an ORP sensor;
adding a stabilizer and a chlorine source to the coating slurry;
mixing the stabilizer and the chlorine source in the slurry to form a stabilized chlorine composition; and
adding additional stabilizer, chlorine source, and/or stabilized chlorine to the coating slurry and/or the component thereof if the measured first pH and/or first ORP decreases.
